(19) United States
(12) Patent Application Publication
(10) Pub. No.: US 2007/0218491 A1
(43) Pub. Date: Sep. 20, 2007

(54) Oligomerization of amyloid proteins

(75) Inventors: Sara Vasan, New York, NY (US); James G. Moe, New York, NY (US); Eliot Davidowitz, West Hempstead, NY (US); Pedro M. Rojas, Corona, NY (US); Abraham Grossman, Pleasantville, NY (US)

Correspondence Address: DAVIDSON, DAVIDSON & KAPPEL, LLC, 485 SEVENTH AVENUE, 14TH FLOOR, NEW YORK, NY, 10018, US

(73) Assignee: Oligomerix, Inc., New York, NY (US)

(21) Appl. No.: 11/704,079

(22) Filed: Feb. 8, 2007

Related U.S. Application Data
(60) Provisional application No. 60/771,364, filed on Feb. 8, 2006.
(60) Provisional application No. 60/861,899, filed on Nov. 30, 2006.
(60) Provisional application No. 60/859,656, filed on Nov. 17, 2006.
(60) Provisional application No. 60/851,586, filed on Oct. 13, 2006.
(60) Provisional application No. 60/790,740, filed on Apr. 10, 2006.

(51) Int. Cl. C12Q 1/68; G01N 33/53; A61K 31/727

(52) U.S. Cl. 435/6; 435/7.1; 514/56

(57) ABSTRACT

The present invention provides methods and, accordingly, in vitro systems for generating stable and soluble oligomers of amyloid proteins, under physiological pH and temperature; and hence, a system for identifying and validating drugs that have the potential to prevent formation of soluble oligomers of amyloid proteins, to disaggregate soluble oligomers of amyloid proteins already formed and possibly disaggregate downstream larger insoluble aggregates of amyloid proteins.

### RELATED APPLICATIONS

This application claims priority to Provisional Application Ser. No., 60/771,364, filed Feb. 8, 2006; Provisional Application Ser. No. 60/790,740, filed Apr. 10, 2006; Provisional Application Ser. No. 60/851,586, filed Oct. 13, 2006; Provisional Application Ser. No. 60/859,656, filed Nov. 17, 2006; and Provisional Application Ser. No. 60/861,899, filed Nov. 30, 2006, the disclosures of which are hereby incorporated by reference in their entireties.

### BACKGROUND OF THE INVENTION

Protein misfolding and aggregation are critical steps in the pathogenesis of a number of neurodegenerative diseases, including amyloid diseases such as Alzheimer's disease (AD). AD pathogenesis is characterized by the aggregation of misfolded neuronal proteins into soluble and insoluble intracellular and extracellular aggregates within the brain. Two histopathological hallmarks are found in brains of AD patients: neurofibrillary tangles (NFTs) and amyloid plaques (APs). APs are primarily composed of beta amyloid peptide, whereas NFTs are formed as intraneuronal structures composed primarily of filaments of hyperphosphorylated isoforms of the microtubule (“MT”) associated protein tau.

Tau protein is normally found in greatest abundance in the axon where it helps stabilize the cytoskeleton, but during neurodegenerative disease tau becomes hyperphosphorylated and dissociates from microtubules, accumulates in the cytosol and assembles into soluble and insoluble aggregates. Neuronal degeneration leads to release of intracellular tau into the extracellular environment where it may have cytotoxic effects on neighboring cells (Gomez-Ramos et al., FEBS Letters, 580: 4842-4850 (2006)). Loss of tau function causes destabilization of MT structure and disrupts neuronal transport and neural circuits, eventually leading to neuronal death. Additional mechanisms have been proposed to explain the formation and toxicity of tau protein aggregates including aberrant cell-cycle activation (Andorfer C., et al., J. Neurosci. 25:5446-5454 (2005); Khurana et al., Curr. Biol. 16:230-241 (2006)), hyperphosphorylation, sequestration and loss of tau function (Iqbal et al., Acta Neuropathol 109: 25-31 (2005)), and an imbalance of tau isoforms causing dysregulation of microtubule dynamics (Ginsberg et al., J. Neurochem. 96(5):1401-8 (2006)).

Over-expression of tau in various animal models has been shown to induce neurodegeneration (Andorfer et al., J. Neurosci. 25: 5446-5454 (2005); Ramsden M., et al., J. Neurosci. 25(46): 10637-10647 (2005)). Others have observed that neuronal cell loss occurs disproportionately to the number of NFTs in AD (Krill J. J., et al., Acta Neuropathol (Berl) 103(4):370-376(2002)). Neuronal loss in the absence of NFTs in a tau-over-expressing Drosophila model has also been reported (Wittmann et al., Science 293:711-714 (2001)), suggesting that the events that lead from tau accumulation to neurodegeneration may not involve filament formation. Repression of expression of mutant tau in transgenic mice led to improved memory and decreased neuronal cell loss even though filaments continued to form, demonstrating that filament formation is not the underlying cause of neuronal cell loss (Santacruz et al., Science 309 (5733): 476-81 (2005)). In a mouse model for AD overexpressing Aβ and mutant tau proteins, reduction of both soluble Aβ and soluble tau was necessary for effective inhibition of cognitive decline (Oddo et al., J. of Bio. Chem. 281(51):39413-39423 (2006))—further supporting the neurotoxic role of the soluble species of amyloid proteins in neurodegenerative disease. Most recently, hippocampal synaptic pathology and microgliosis were shown to be the earliest manifestations of neurodegenerative tauopathies months before the development of tangles in the P301S tauopathy mouse model (Yoshiyama, Y., et al., Neuron 53(3):337-351 (2007)).

The primary neurotoxic structure of Aβ is soluble oligomers. Recent reviews on this theory include: Catalano S. M. et al., Curr Top Med Chem 6(6):597-608 (2006) and Walsh D M, et al., Biochem Soc Trans 33(Pt 5):1087-90 (2005).

Additional work based on the development of polyclonal antibody A11 led to the general hypothesis that because soluble amyloid oligomers have a common structure they also have a common mechanism for causing disease (Kayed et al., Science 300:486-491(2003)). However, this work did not present evidence that demonstrated that tau oligomers have this structure or have neurotoxicity. A subsequent publication has shown that soluble tau containing tau oligomers applied to the extracellular environment of cultured neuroblastoma cells are more neurotoxic than insoluble tau aggregates and that muscarinic receptors may mediate this effect (Gomez-Ramos et al., FEBS Letters 580:4842-4850 (2006)). The NMDA receptor was also shown to be involved in tau-induced neurotoxicity further indicating that disruption of tau interaction with receptors may have therapeutic value (Amadoro G. et al., Proc. Natl. Acad. Sci. USA 103(8):2892-7 (2006)).

Hence there remains a need for methods and strategies targeted at detecting, understanding and treating amyloid diseases, with specific concentration on the role that soluble oligomers of amyloid proteins play in the disease pathology.

### OBJECTS AND SUMMARY OF INVENTION

A strategy for the study and treatment of amyloid disease, including AD, is to inhibit the accumulation of soluble oligomers of amyloid proteins by inhibiting their formation and/or their disaggregation. In vitro methods to generate soluble tau oligomers analogous to those found in AD, for example, will provide a model for studying the pathology of tauopathies and simplify the identification of drugs that decrease the soluble tau oligomer burden (and, accordingly, represent potential new drug targets). Thus, the subject of this application is the fast and efficient generation of stable soluble oligomers of amyloid proteins for use in modeling amyloid diseases and designing assays for the evaluation and testing of potential new drug targets for amyloid diseases.

It is an object of the present invention to provide soluble oligomers of a cationic amyloid protein and methods of producing soluble oligomers of a cationic amyloid protein.

It is an object of certain embodiments of the present invention to provide a method of mimicking part of a pathway of an amyloid protein disease comprising incubating a mixture of an effective amount of a cationic amyloid protein and an effective amount of a polyanion, under physiological conditions, for a time period of about 10 minutes to about 120 minutes, to generate a stable soluble oligomer of the cationic amyloid protein that is indicative of the amyloid protein disease.

It is an object of certain embodiments of the present invention to provide a method for evaluating the ability of a therapeutic to affect an amyloid disease by (i) incubating a mixture of a cationic amyloid protein, a polyanion, under physiological conditions; (ii) applying a therapeutic to be evaluated to the mixture; (iii) analyzing the mixture to detect the presence or absence of a soluble oligomer of the cationic amyloid protein; whereby the absence of a soluble oligomer of the cationic amyloid protein indicates the therapeutic is able to inhibit oligomer formation or disaggregate oligomer of the cationic amyloid protein, thereby indicating potential ability of the therapeutic to affect an amyloid disease.

It is an object of certain embodiments of the present invention to provide a method for identifying a therapeutic which modulates the formation of soluble tau oligomers by (i) incubating a mixture of a tau isoform and a polyanion under physiological conditions; (ii) applying a therapeutic; (iii) analyzing the mixture to detect the presence or absence of a soluble tau oligomer; (iv) correlating the presence or absence of a soluble tau oligomer in the mixture to modulation of the formation of soluble tau oligomers by the therapeutic, whereby the absence of soluble tau oligomer indicates the therapeutic modulates the formation of soluble tau oligomers.

It is an object of certain embodiments of the present invention to provide a method of preparing a soluble tau oligomer that is stable under physiological conditions comprising incubating a mixture of a tau isoform with a polyanion, in a ratio from about 2:1 to about 20:1 of the tau isoform to the polyanion, under physiological conditions for about 30 minutes to about 60 minutes.

It is an object of certain embodiments of the present invention to provide a method of generating a stable, soluble oligomer of a cationic amyloid protein of a specified number of subunits by (i) choosing a polyanion of appropriate size and charge distribution; (ii) mixing an effective amount of the polyanion with an amount of an effective amount of a cationic amyloid protein to create a mixture under physiological conditions; (iii) allowing sufficient time for the polyanion and cationic amyloid protein to form stable and soluble oligomers of the cationic amyloid protein of specified number of subunits; such that the effective amounts of the polyanion and cationic amyloid protein are determined so that the cationic amyloid protein will be completely depleted after allowing sufficient time for formation of the stable and soluble oligomers, thereby trapping the stable and soluble oligomers at the specified number of subunits due to the size and charge distribution of the chosen polyanion.

Other objects and advantages of the present invention will become apparent from the disclosure herein.

As used herein, the term cationic protein refers to a protein that is cationic, i.e., having a positive charge, at or near physiological pH.

### BRIEF DESCRIPTION OF THE FIGURES

FIG. 1 illustrates the theory underlying the invention, modeling the polyanion-mediated oligomer formation of a cationic amyloid protein, in this case tau.

FIG. 2 depicts oligomer characterization by size exclusion chromatography. Sephacryl S-300 column (1×31 cm) was equilibrated with PBS and calibrated using high molecular weight standards from Amersham. Reaction products of a control incubation of tau441 without RNA (A); tau441 incubated with RQ11+12 (B); and tau441 incubated with RQT157 (C); were applied to the column, eluted with PBS and analyzed using modified ELISA.

FIG. 3 depicts oligomer profile after size exclusion chromatography of, tau412 incubated with RNA polyanion and tau412 incubated without RNA polyanion.

FIG. 4 depicts the time course of oligomer formation using modified ELISA to measure the oligomers. The formation of oligomers is rapid, reaching an optimum within an hour of incubation of tau412 with an RNA polyanion (TRQ), and tau412 incubated with reaction buffer.

FIG. 5 depicts oligomer formation validated using A11 antibody specific for oligomer conformation (A11): amplified ELISA.

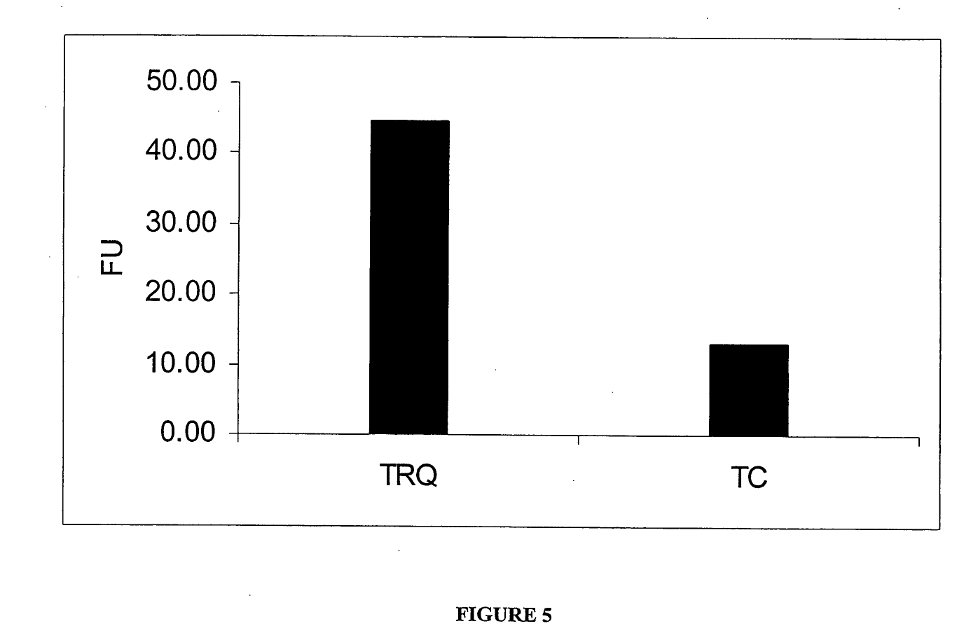

FIG. 6 depicts how tau352 (A) and tau412 (B) generate nucleoprotein complexes (NPC) with RNA that are stable in 7 M Urea. The products of tau352 and Tau412 interaction with RNA were analyzed using 7 M PAGE and stained with Cybergold intercalating dye specific for RNA. Asterisks indicate NPC-associated RNA. Product of tau interaction with RQ11+12, MNV, pool of total mRNA and AP, are shown in the lanes 2, 4, 6 and 8 correspondingly. Lanes 1, 3, 5 and 7 represent respective RNAs alone. MW is molecular weight marker. The results indicate that the complexes were stable even after exposure to the urea.

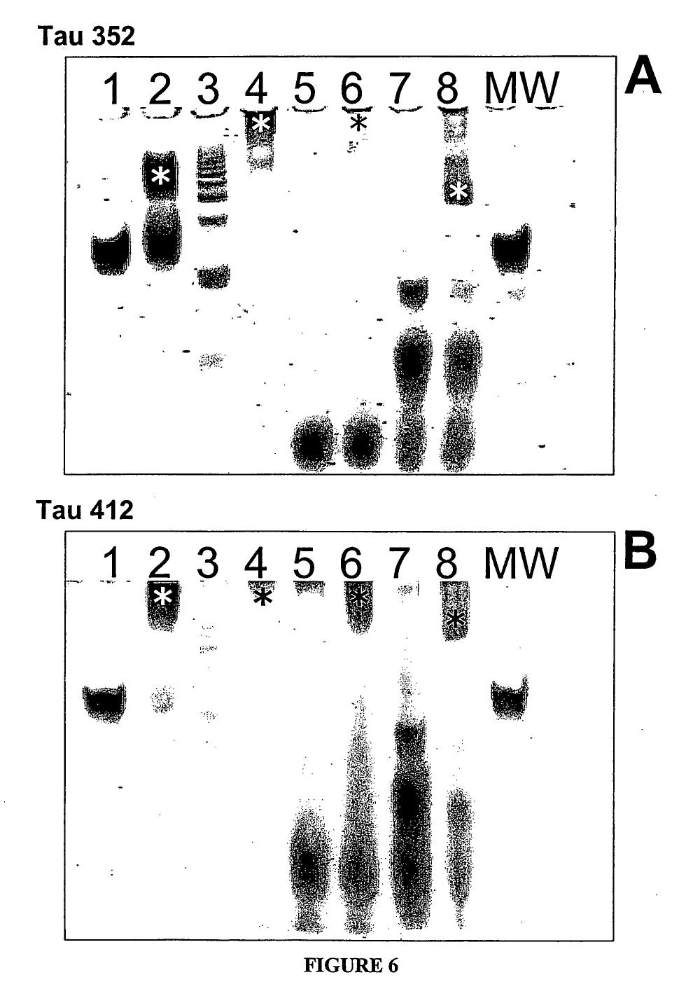

FIG. 7 depicts a schematic diagram comparing the standard ELISA format with the modified ELISA format which may be used in the present invention to detect soluble tau oligomers.

FIG. 8 depicts a graph of the optimization of the molar ratio of tau:RNA (for an RNA of this length and composition), using RQ11+12. Oligomer formation appears optimal with a 5:1 tau:RNA ratio. Higher levels of RNA may inhibit the formation of oligomers that can be detected using the present capture/reporter antibodies.

FIG. 9 depicts a graph of the sensitivity enhancement for oligomer detection using A11 antibody.

FIG. 10 depicts the time course of oligomer formation using oligomer-specific antibody A11 ELISA to measure the oligomers. pAb A11 is specific for oligomer conformation and recognizes RNA-induced soluble tau oligomers. The formation of oligomers is rapid, reaching an optimum within 30 minutes of incubation of tau412 with an RNA polyanion (TRQ), and tau412 incubated with reaction buffer.

FIG. 11 depicts identification of soluble tau oligomers in human CSF samples showing the ability of the pAb A11 ELISA of the present invention to detect both soluble tau oligomers generated in vitro, and soluble tau oligomers from biological specimens.

FIG. 12 depicts tau412 oligomer formation with various RNAs, detected with A11 Ab.

FIG. 13 depicts a graph comparison of tau oligomer formation when incubated with RNA and with heparin at various ratios.

FIG. 14 depicts a graph showing the inhibition of RNA-facilitated tau oligomer formation by curcumin. Grey bars represent incubations including RQ11+12 RNA polyanion; black bars represent incubations without the RNA polyanion.

FIG. 15 depicts an ELISA analysis of oligomers formed with RQT157 RNA at different molar ratios with tau 412.

FIG. 16 depicts modified ELISA results for DNA polyanion-facilitated oligomer formation with tau 412. The columns from left to right are: tau 412 negative control (no RNA); tau 412 incubated with RQ11+12 (positive control); tau 412 incubated with sheared salmon sperm DNA in 1:1 ratio; tau 412 incubated with sheared salmon sperm DNA in 5:1 ratio; tau 412 incubated with sheared calf thymus DNA in 5:1 ratio; tau 412 incubated with poly dT DNA (˜700 bases) in 1:1 ratio; and tau 412 incubated with poly dT DNA (˜700 bases) in 5:1 ratio.

### DETAILED DESCRIPTION

Amyloid diseases are typically characterized by the deposition of amyloid proteins in various organs of the body. It is generally believed that amyloid diseases share common pathogenic mechanisms that eventually lead to protein fibril formation and deposition. While older theories attributed the pathology of amyloid diseases to these insoluble deposits of amyloid proteins, recent discoveries suggest that such deposits (such as NFTs and APs) may be a downstream symptom of an amyloid disease or even a defensive mechanism as opposed to the causal agent. For example, as mentioned above, more recent studies concerning tau, suggest that soluble tau oligomers may be a causal agent; or at a minimum, formation of soluble tau oligomers may represent an earlier step in the disease progression.

The family of amyloid diseases, as well as the proteins associated with those diseases, is known and includes: neurological diseases such as Alzheimer'S (AD), Huntington'S, Parkinson'S, Congophilic Angiopathy and Transmissible Spongiform Encephalopathies (TSEs), like Creutzfeldt-Jakob'S, Bovine Spongiform Encephalopathy, Kuru, and Fatal Familial Insomnia. Amyloid diseases also encompass diabetes mellitus type 2, cardiac amyloidosis, some respiratory disorders such as cystic fibrosis, ocular maladies like cataracts and inclusion body myositis. The associated amyloid proteins include: amylin (IAPP), Huntington, Prion (PrP), tau, alpha-synuclein, amyloid beta and other proteins that will be appreciated by those of skill in the art.

The present invention is directed to the fast and efficient formation of stable, soluble oligomers of cationic amyloid proteins, under physiological conditions. In particular, using an appropriately chosen polyanion molecule, a soluble oligomer of desired size may be formed using the methods and teachings of this invention. This has significant implications towards discovering which size oligomer (i.e., trimer, hexamer, octamer, dodecamer, etc.) is neurotoxic and/or otherwise involved in amyloid disease pathogenesis.

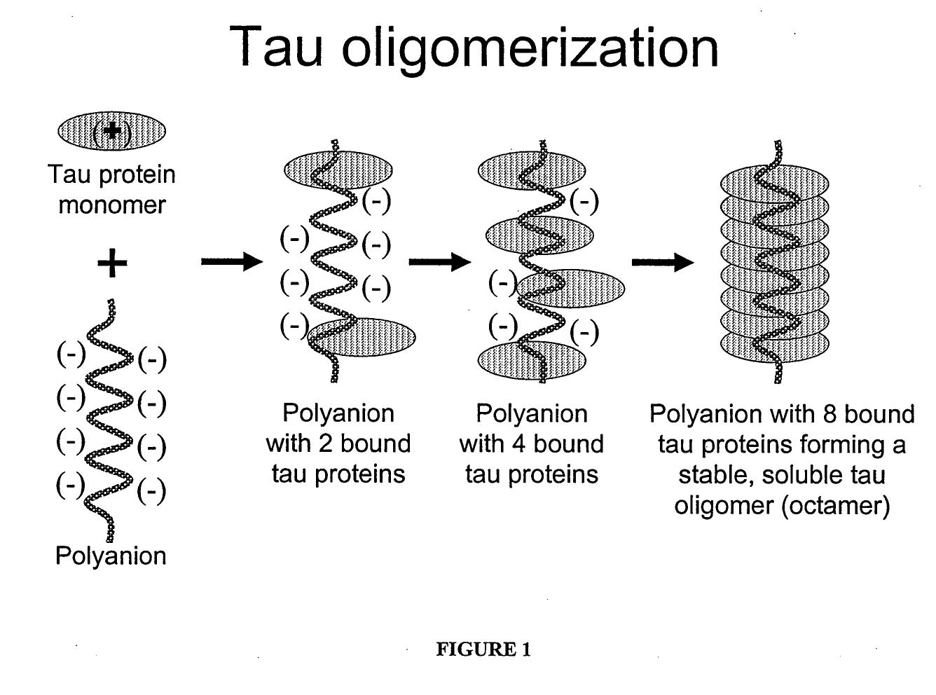

Using effective amounts of a polyanion and low concentrations of a cationic amyloid protein, the protein may be “trapped” by the polyanion (the positive charge of the protein is drawn to the negatively charged polyanion via ionic interactions) which then facilitates the formation of soluble oligomers. (See FIG. 1). The soluble oligomers can then be used as a platform for screening compounds that may inhibit pathological amyloid protein oligomer formation, and thus have great potential for use as an amyloid disease therapeutic. In order to obtain the desired sized oligomers (e.g., a hexamer instead of a dodecamer), the concentration, stoichiometry, size, structure and charge distribution of the polyanion is important. For example, a polyanion that is too large in structure will not bring the protein monomers, at a relatively low concentration, within proximity of each other to facilitate oligomerization. Furthermore, the same effect is achieved by the addition of too much polyanion in relation to the concentration of protein. Conversely, too much protein in relation to a given polyanion may result in the formation of insoluble aggregates of the protein, i.e., driving the reaction too far. Hence, one begins to appreciate the “trapping” concept, where the polyanion acts as a “molecular scaffold” to attract and trap the protein monomers in such a fashion as to facilitate their oligomerization; a larger “scaffold” resulting in a larger oligomer, a smaller “scaffold” resulting in smaller oligomers (see FIG. 1). The fast reaction kinetics observed may be catalyzed by the polyanion scaffold at low protein concentrations by virtue of the fact that the effective concentration of bound tau is much higher than the concentration of free, unbound tau. Because of the speed and efficiency of the methods of the present invention, also included within the scope of the invention is an effective means for researching, diagnosing and screening therapeutics. Such therapeutics include, for example, chemical compounds, drugs, vaccines, methodologies and any other method and/or composition that may have an affect on soluble oligomers and hence, disease pathology. Thus, also included within the scope of this invention, for example, is a high-throughput assay for screening chemical libraries and developing lead optimization studies for compounds for treatment of amyloid diseases such as AD.

The size of the oligomer formed is dependent on the length of the polyanion polymer and its charge distribution. In preferred embodiments the soluble oligomers formed comprise from about 3 to about 20 subunits of protein.

The present invention provides methods and, accordingly, in vitro systems for generating stable and soluble oligomers of amyloid proteins, under physiological pH and temperature; and hence, a system for identifying and validating drugs or methodologies that have the potential to prevent formation of soluble oligomers of amyloid proteins, to disaggregate soluble oligomers of amyloid proteins already formed and possibly disaggregate downstream larger insoluble aggregates of amyloid proteins. Included in the invention is a 96-well format system, which provides a platform for screening chemical libraries and developing lead optimization studies for compounds for Alzheimer'S disease and other amyloid diseases. This format is easily scaleable to a high throughput format for drug screening (i.e., a 384-well, automated assay).

Generally, polyanions facilitating tau oligomer formation should bind multiple tau molecules via salt bridges, resulting in bringing relatively low concentrations of tau in solution to a relatively high local concentration when bound to the polyanion scaffold. Furthermore, it is presumed that the polymer flexibility and charge distribution should be such that bound tau molecules are in close proximity to each other so that the tau molecules can interact and form stable oligomers. Polyanions may be more effective if they are flexible to allow more freedom for tau-tau interaction. Spacing of charge and charge density should maximize tau-tau interactions. The polyanions may be linear, circular, or branched. They may be used free in solution, conjugated to other soluble molecules or attached to solid surfaces or beads.

The system of the present invention uses a polyanion molecule for rapidly enabling soluble oligomer formation and stabilization at low tau concentration at physiological pH and temperature. Tau and prion are examples of cationic amyloid proteins; included within the scope of this invention are cationic amyloid proteins, such as tau and prion, as well as fragments and derivatives thereof. Polyanion molecules behave as a “molecular scaffold” for the cationic proteins binding them so that they are in close association with each other to favor oligomer formation. Thus, as mentioned above, factors such as charge, size, structure, concentration and stoichiometry are key considerations for selecting an appropriate polyanion scaffold—i.e., one that will create a stable soluble oligomer, but will not drive the reaction past that point towards formation of larger, insoluble protein aggregates. Furthermore, by controlling the size of the polymer and hence its charge, the size of the oligomer trapping can be controlled such that oligomers of various sizes can be generated, isolated and characterized. For example a small polyanion that could only bind a small number of tau molecules (e.g., four) would need a lower ratio of tau:polymer than a large polyanion that could bind significantly more (e.g., 10-20) tau molecules—e.g., a 4:1 ratio of tau:polymer vs. a 10:1 ratio of tau:polymer. The ability to control the ultimate size of the tau oligomer is therefore determined both by the length and charge distribution of the polymer used, by the ratio of tau:polyanion, and by the concentration of tau protein such that as the reaction nears completion, the unbound tau has been depleted to such a low concentration that the probability of it interacting with the oligomer structures that have formed becomes vanishingly low. Thus upon choosing an appropriate polyanion, two parameters that will influence the choice of the ratios used are 1) size or length of the polymer and 2) charge distribution. For example, for oligomer formation using a polymer of about the same size as RQ11+12 that has a similar charge distribution, it is expected that a 5:1 (tau:polyanion) ratio would be preferable. However, for a smaller RNA polyanion or a synthetic polymer with a low charge density similar to heparin, a lower ratio like, for example, 1:1 (tau:polyanion) may be more preferable. (See examples below). Furthermore, polymers with disperse negative charges would require a more flexible backbone (i.e. not highly conjugated), thus it is unlikely that a polystyrene-like polymer with a charge distribution similar to heparin would be useful as a molecular scaffold for oligomer formation.

Using tau and naturally occurring isoforms and derivatives thereof as an example, the formation of soluble oligomers was done using RNAs, DNAs, naturally occurring polyanions and synthetic polyanions. Specifically, nucleic acids having a size of about 10 nucleotides to about 10,000 nucleotides were used; this is described in more detail in the Examples below. Stable and soluble oligomers were formed using RNAs such as RQ11+12, RQT157 and MNV, as well as DNAs and heparin (see Examples below). The reactions were performed under physiological conditions, and most preferably at neutral pH (around 7.4) and temperature of around 37° C., thus eliminating the effects of high temperature or pH on the stability of the test compounds. The DNAs used were sheared genomic DNA from salmon sperm and calf thymus, as well as, synthetic single strand poly dT (˜700 bases).

In addition to the simplicity and speed at which the assay is performed, it requires low concentrations of oligomer-forming protein. This is a significant advantage given the high cost of recombinant protein that is necessary using traditional methods for large scale screening of chemical libraries. Furthermore, the reaction is rapid, going to completion in 10 to 120 minutes, and preferably 30-60 minutes, which is a significant advantage for large-scale screening.

The formation of soluble tau oligomers was confirmed using various immunoassay techniques, which included the use of various antibodies to confirm not only tau-specific oligomers, but formation of oligomers generally. Formation of oligomers was also confirmed using size exclusion chromatography. Results have consistently shown distinct peak(s) using size exclusion chromatography, demonstrating the formation of a single species as opposed to several species of differently sized oligomers, providing proof of trapping of tau oligomers of a defined size. The presently described methods for rapidly generating and detecting oligomers facilitates simple, sensitive high throughput screening to identify compounds that can inhibit the pathological process of protein-misfolding diseases such as tauopathies and amyloid diseases.

Furthermore, the oligomers are amenable for isolation and purification by gel filtration; thus providing a tool to screen and identify compounds that can disrupt pre-formed oligomer structure.

In certain embodiments, the present invention provides a soluble oligomer of a cationic amyloid protein formed by incubating a mixture of an effective amount of a cationic amyloid protein and an effective amount of a polyanion, under physiological conditions, for a time period of about 10 minutes to about 120 minutes, to generate a stable, soluble oligomer of the cationic amyloid protein that is indicative of the amyloid protein disease, and methods of generating the soluble oligomer, thereby mimicking part of the pathway of an amyloid protein disease.

In certain embodiments, the present invention is directed to a method for evaluating the ability of a therapeutic to affect an amyloid disease by (i) incubating a mixture of a cationic amyloid protein, a polyanion, under physiological conditions; (ii) applying a therapeutic to be evaluated to the mixture; (iii) analyzing the mixture to detect the presence or absence of a soluble oligomer of the cationic amyloid protein; whereby the absence of a soluble oligomer of the cationic amyloid protein indicates the therapeutic is able to inhibit oligomer formation or disaggregate oligomer of the cationic amyloid protein, thereby indicating potential ability of the therapeutic to affect an amyloid disease.

In certain embodiments, the present invention is directed to a method of generating a stable, soluble oligomer of a cationic amyloid protein of a specified number of subunits by (i) choosing a polyanion of appropriate size and charge distribution; (ii) mixing an effective amount of the polyanion with an effective amount of a cationic amyloid protein to create a mixture under physiological conditions; (iii) allowing sufficient time for the polyanion and cationic amyloid protein to form stable and soluble oligomers of the cationic amyloid protein of specified number of subunits; such that the effective amounts of the polyanion and cationic amyloid protein are determined so that the cationic amyloid protein will be completely depleted after allowing sufficient time for formation of the stable and soluble oligomers, thereby trapping the stable and soluble oligomers at the specified number of subunits due to the size and charge distribution of the chosen polyanion.

In certain embodiments, the soluble oligomer generated by the above methods can be used to generate an antibody. In preferred embodiments, the antibody is a monoclonal antibody.

In preferred embodiments, the time period is about 30 to about 60 minutes, and the physiological conditions include a temperature of about 37° C. and a pH of about 7.4.

In certain embodiments, the amount of the cationic amyloid protein is from about 40 nM to about 4 μM, and the cationic amyloid protein is a tau protein.

In further embodiments, the polyanion is an RNA having a length of from about 10 nucleotides to about 10,000 nucleotides; in preferred embodiments, the RNA is RQ11+12. In other embodiments, the polyanion is a DNA having a length of from about 10 nucleotides to about 10,000 nucleotides and is single stranded or double stranded.

In certain embodiments of the present invention, the mixture comprises a ratio of from about 2:1 to about 20:1 of tau protein to the RNA.

In certain embodiments of the present invention, the soluble oligomer comprises about 3 to about 20 subunits of the cationic amyloid protein.

In preferred embodiments of the present invention, the polyanion has a molecular weight of about 2,000 Daltons to about 125,000 Daltons, and is a proteoglycan, such as heparin, or a sulfated polysaccharide.

In other embodiments of the present invention, the polyanion is a synthetic polyanion, and has a linear structure, a branched structure or a circular structure.

In certain embodiments, the mixture is analyzed using an immunological assay. In preferred embodiment, the immunological assay is a standard ELISA, or a modified ELISA that uses the same antibody to capture and to report the soluble oligomer of the cationic amyloid protein.

In preferred embodiments, an A11 or a DC11 antibody is used in the immunological assay.

In certain embodiments, the compound evaluated is used for an assay for evaluating the ability of the compound to affect amyloid diseases.

In other embodiments, the present invention is directed to a method for identifying a therapeutic which modulates the formation of soluble tau oligomers by (i) incubating a mixture of a tau isoform and a polyanion under physiological conditions; (ii) applying a therapeutic to the mixture; (iii) analyzing the mixture to detect the presence or absence of a soluble tau oligomer; (iv) correlating the presence or absence of a soluble tau oligomer in the mixture to modulation of the formation of soluble tau oligomers by the therapeutic, whereby the absence of soluble tau oligomer indicates the therapeutic modulates the formation of soluble tau oligomers.

The invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense. Any reference to any patent or publication made herein is hereby incorporated by reference in its entirety for all purposes.

### EXAMPLES

### Tau Isoforms

The table below lists six tau isoforms along with the corresponding terminology used throughout this application. For convenience, the table also provides alternative means of identifying these isoforms, commonly used by those of skill in the art; thus, “N” refers to the alternatively spliced exons included in the amino-terminal end of tau isoforms and “R” refers to microtubule binding domain repeat units present:

| Variant | | | Terminology |
|---|---|---|---|
| (isoform) | # Residues | Mass (kDa) | Used Herein |
| 0N3R | 352 | 36.8 | tau352 |
| 0N4R | 383 | 40.0 | tau383 |
| 1N3R | 381 | 39.7 | tau381 |
| 1N4R | 412 | 42.9 | tau412 |
| 2N3R | 410 | 42.6 | tau410 |
| 2N4R | 441 | 45.9 | tau441 |

### Example 1

Formation of a Soluble Tau Oligomer with RNA

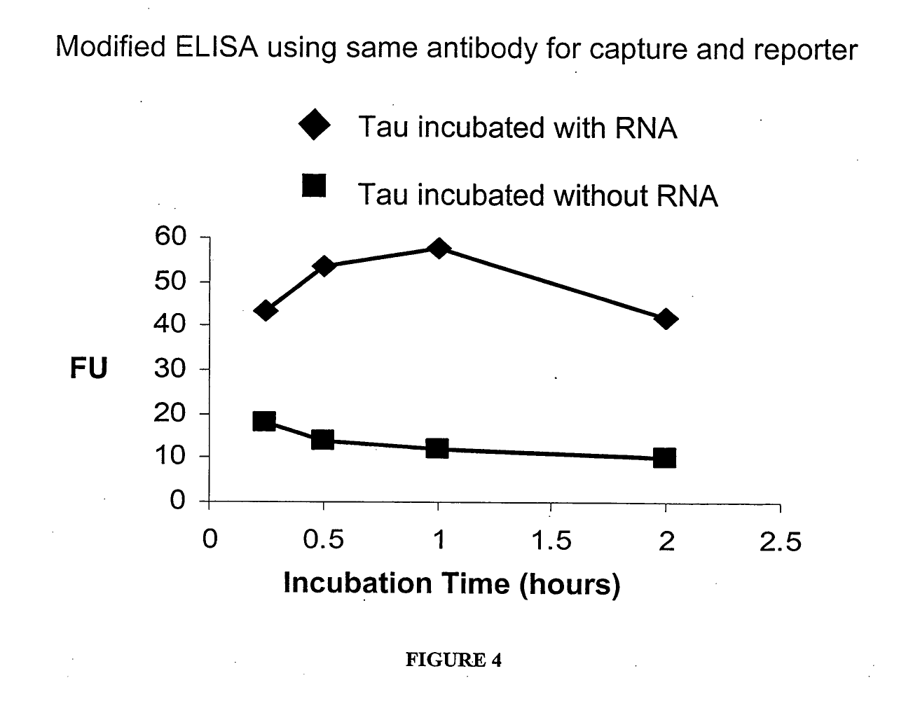

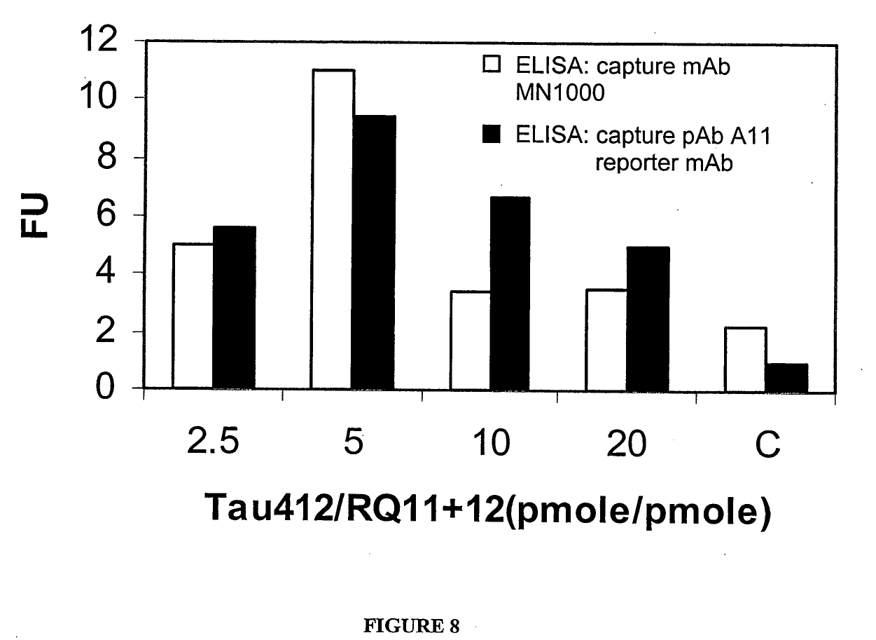

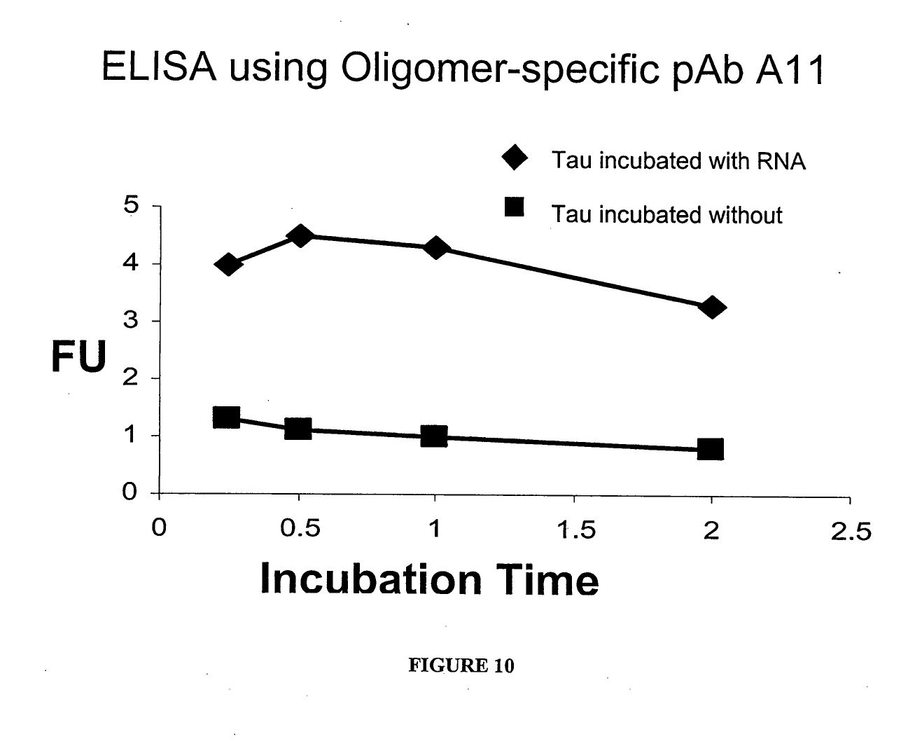

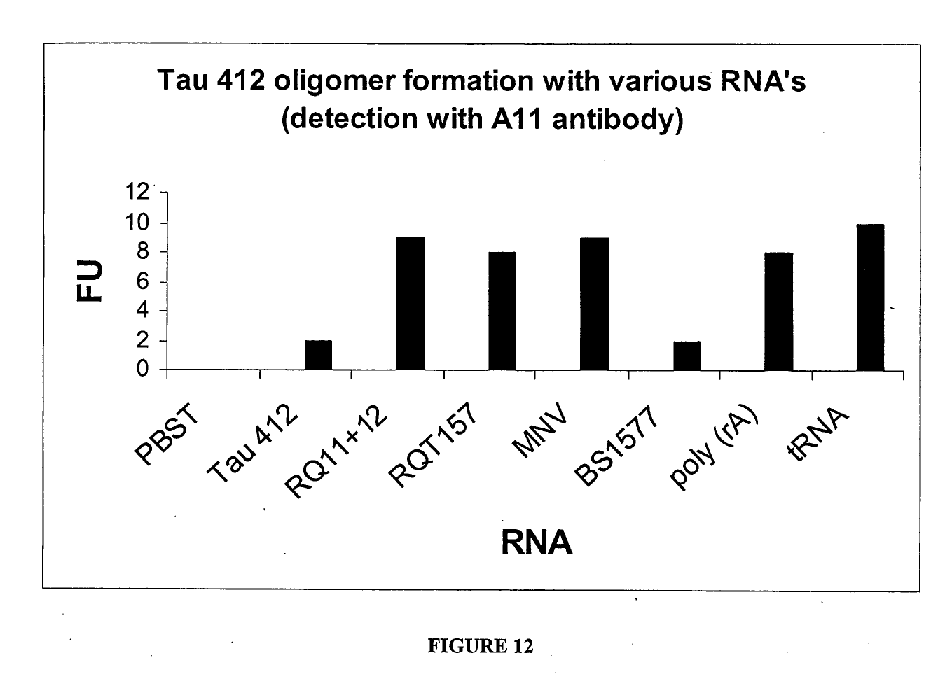

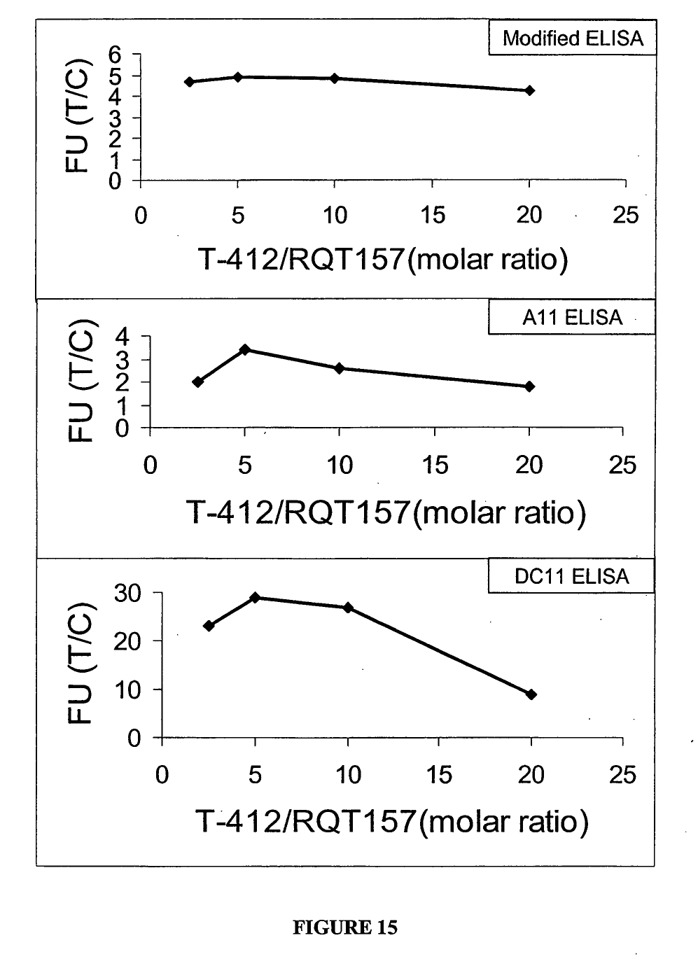

RNA polyanion was used to facilitate oligomer formation of tau protein in vitro using the following, preferred, conditions: 20 pmoles of tau412 was reacted with 4 pmoles of a 197 base RNA, i.e., RQ11+12, in 50 mM Tris-HCl buffer at pH of 7.4 and incubated for 1 hour at 37° C. For oligomer formation, the ratio of tau:RQ11+12 RNA was found to be optimal at a ratio of 5:1 (FIG. 8). The 5:1 ratio was optimal for the RNA RQT157 as well (FIG. 15). Multiple types of RNAs including unstructured polyA (˜700 bases) and highly structured RNAs, tRNA (˜100 bp), RQ11+12 (197 bases), RQT157 (157 bases) and MNV (87 bases) all facilitated tau oligomer formation using the 5:1 tau:RNA ratio (FIG. 12); demonstrating the general nature of RNA facilitation of tau oligomer formation. Note, however, that the RNA polyanion BS1577—a significantly smaller polyanion—did not facilitate oligomer formation at this ratio; demonstrating that size, charge distribution and concentration/stoichiometry all contribute to oligomer formation according to this invention. The kinetics of oligomer formation was rapid. An RNA-facilitated oligomer formation time course was analyzed by three different ELISAs (described below) and showed that oligomers peaked within 0.5-1 hr (FIGS. 4 and 10). Physiological pH and temperature were used in the assay to simulate biological conditions.

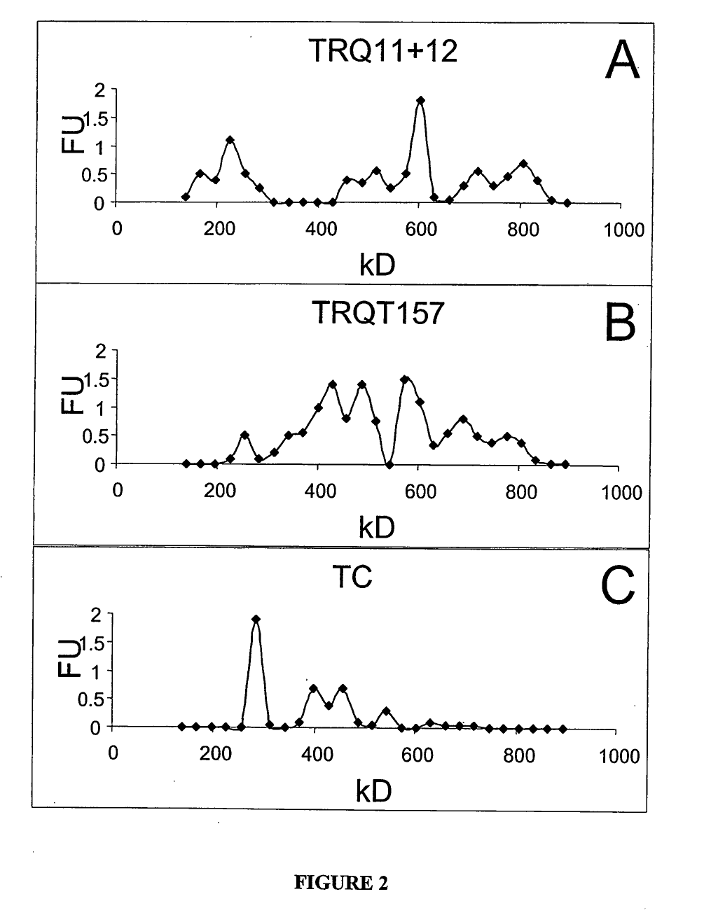

RQ11+12 is approximately 33% larger than RQT157, and generates peaks that are 50% larger (600 kDa versus 400 kDa) and 25% larger (750 kDa versus 600 kDa) than those generated by RQT157 (FIG. 2). Therefore, although the optimal ratio of 5:1 (tau:polyanion) was the same for the different polyanions used in this example, the size of the soluble oligomer formed will vary based on the size of the polyanion used; thus showing that the choice of polyanion can govern the size of the resulting soluble oligomers. This is further exemplified in Example 4 below.

### Example 2

Size Exclusion Chromatography to Estimate Oligomer Size

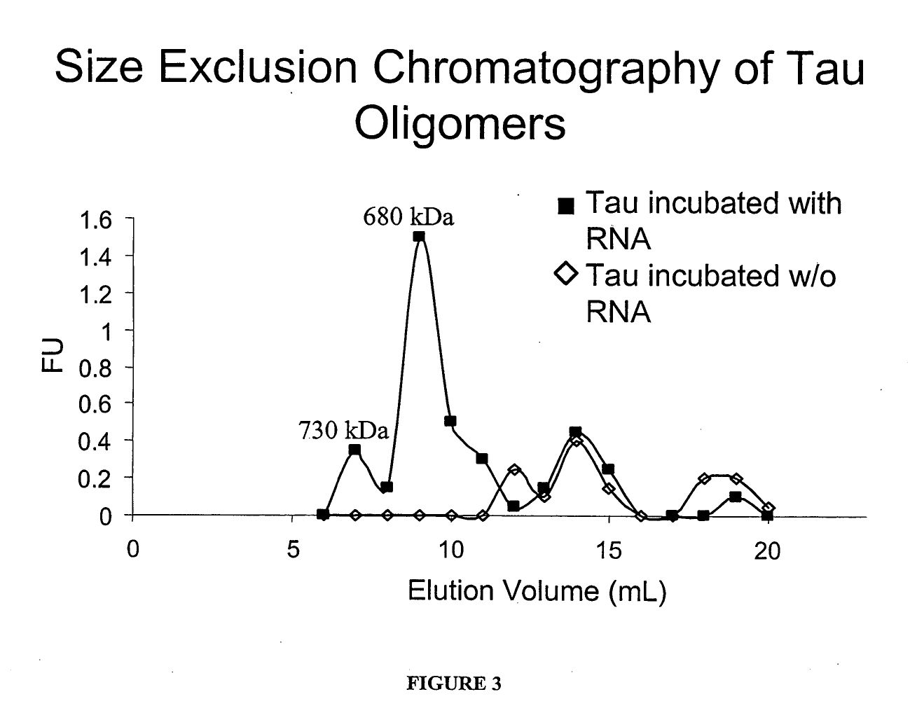

Purification of soluble tau oligomers was performed using size exclusion chromatography on Sephacryl S-300 and detection of tau oligomers was achieved using a modified ELISA (described below). Tau412 protein (42.9 kDa) incubated with RQ11+12 RNA (65 kDa) gave a predominant peak at 680 kDa (FIG. 3) based on elution profile of molecular weight standards thyroglobulin 669 kDa, ferritin 440 kDa, catalase 232 kDa, and aldolase 158 kDa indicating that the predominant oligomer species formed was composed of approximately 15 tau monomers assuming that the oligomer structure did not cause the complex to migrate at an aberrant size within the column. Tau protein has a native unstructured conformation giving it a larger radius than a typical globular protein of the same molecular weight. Thus, this technique is not ideal for sizing tau and its oligomers, but may still be used to separate monomeric tau and different size oligomers. The predominant sharp peak at 680 kDa shows that the oligomer formed is stable over the course of the reaction, i.e., incubation, chromatographic separation and detection (because it accumulates at a particular size), the chromatographic separation, and detection (about two hours) and that the RNA functionally traps this complex and does not allow the tau to further aggregate.

### Example 3

Immunological Analysis of Oligomer Formation

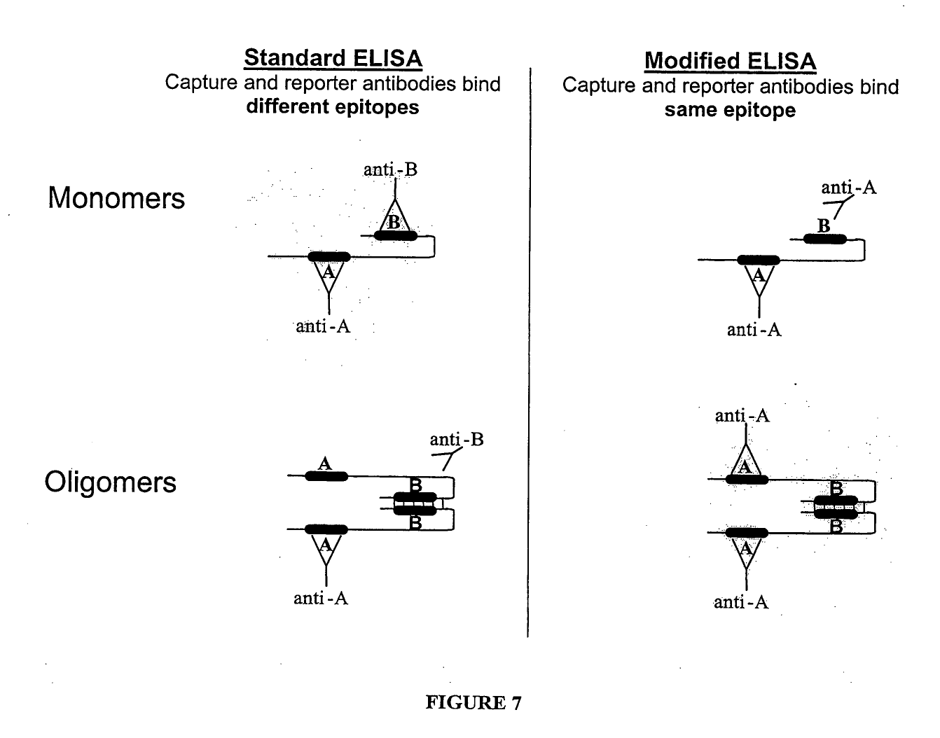

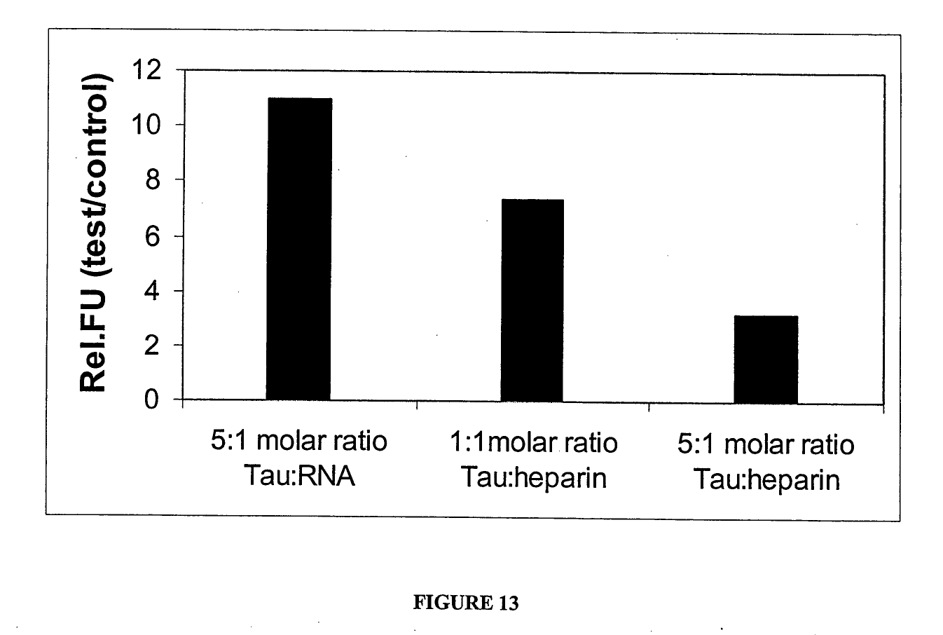

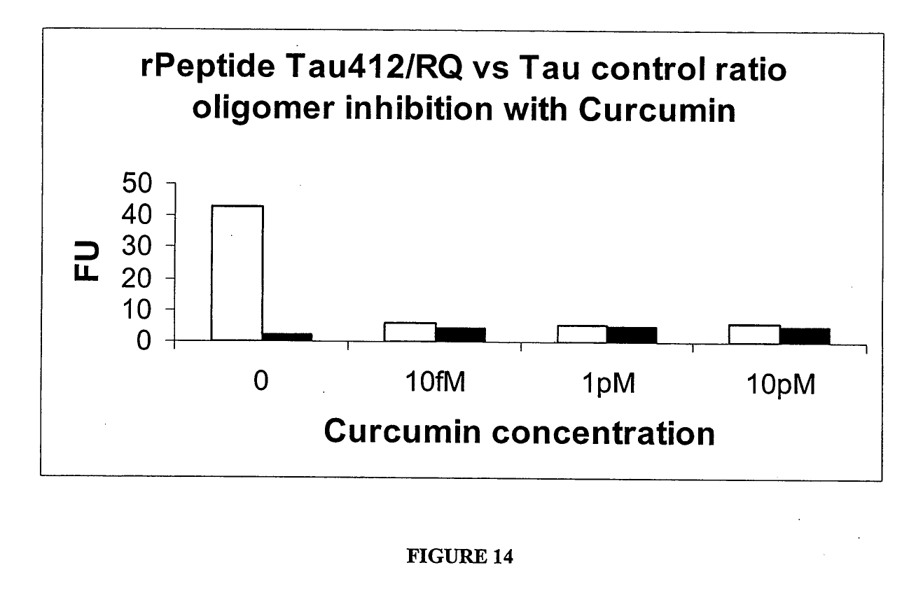

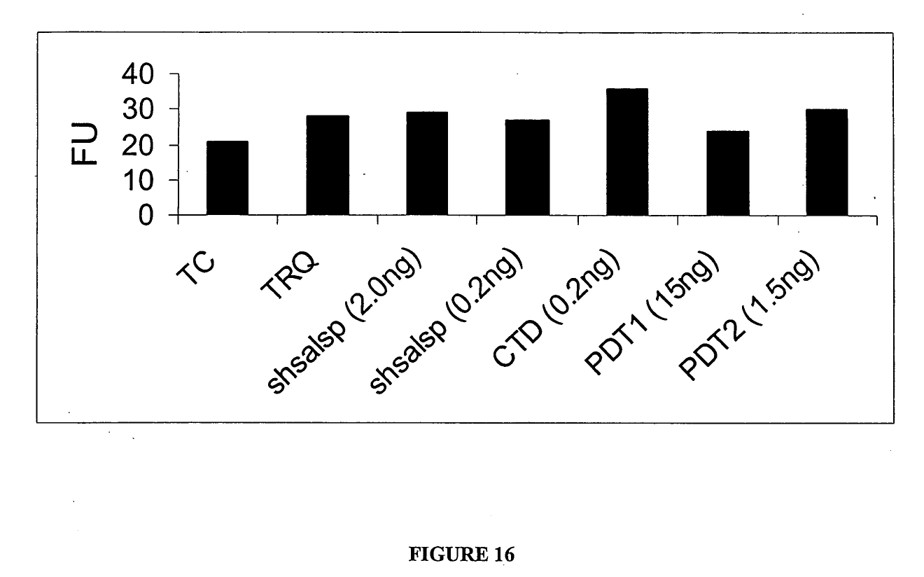

Formation of tau oligomers was analyzed using two different ELISA methods to provide verified data. The “modified” ELISA uses the same monoclonal antibody for capture and detection. Insoluble aggregates are removed in the ELISA procedure during washing steps. Only soluble aggregates of tau, not monomers, are detected with this ELISA because the detection and reporter antibody recognizes an epitope which is present only once within tau. At least one more tau molecule must be present in the complex for detection with the same antibody (method illustrated in FIG. 7). The modified ELISA used is similar to the modified ELISA previously used for identification of Aβ oligomers (Sian et al., Biochem. J. 349(Pt 1):299-308 2000)) and PrP oligomers (Vasan et al., Neurochem Res 31(5):629-37(2006)). This assay used antibody MN1000, recognizing tau epitope amino acids 159-163, for capture and biotinylated MN1000 as reporter. This assay was used to detect tau oligomers in fractions eluted from the size exclusion column (FIG. 3), identify accessible epitopes in tau oligomers (not shown), to follow oligomer formation over time (FIG. 4), to optimize the molar ratio of tau:RNA for oligomer formation (FIGS. 8 and 15), to show heparin and DNA polyanions functions as facilitators of tau oligomerization (FIGS. 13 and 16) to detect soluble tau oligomers in CSF for validation of this target for compound screening and drug development and for screening compounds for inhibition of oligomer formation (FIG. 14).

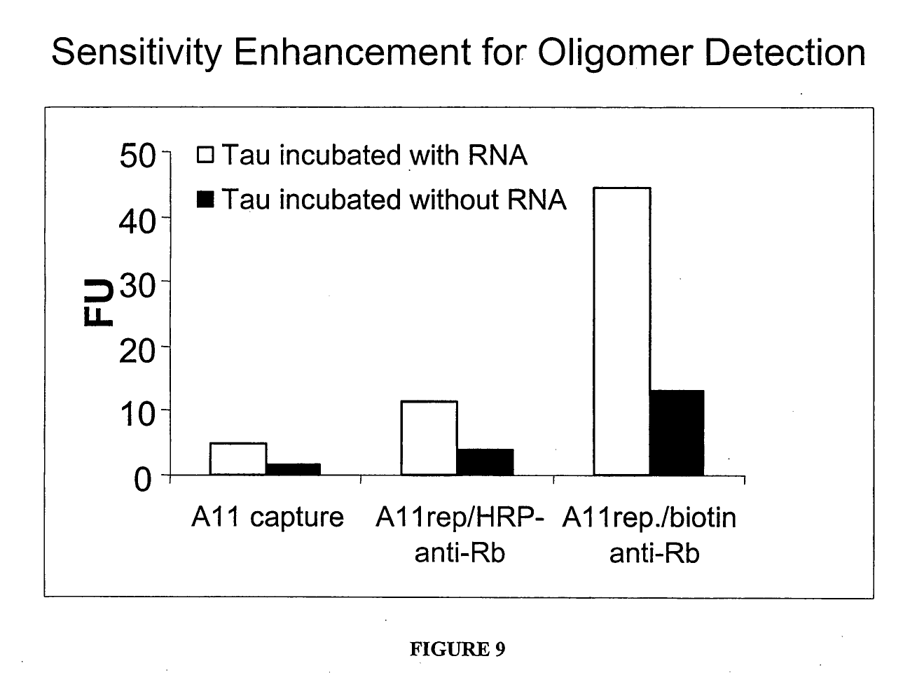

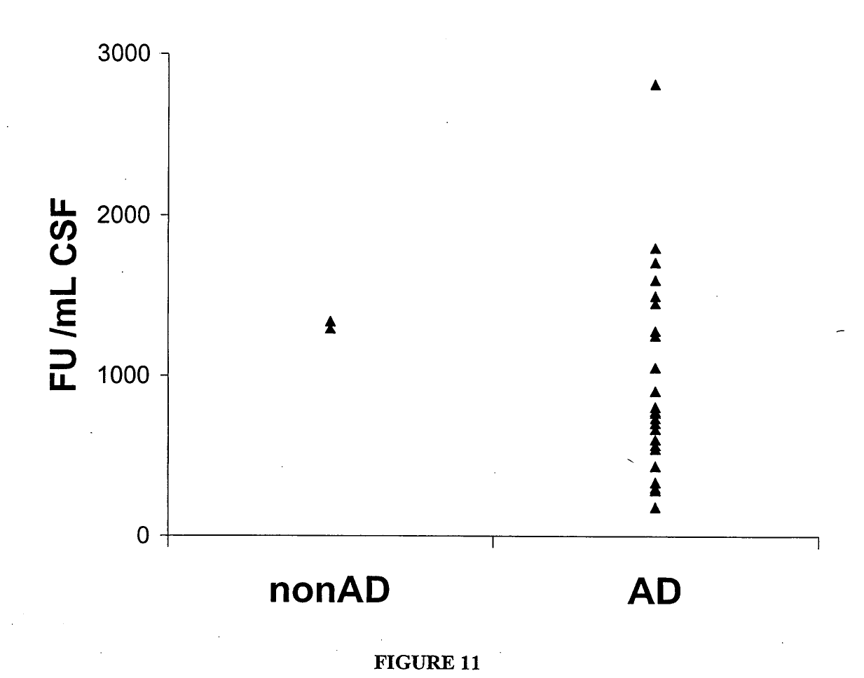

The second ELISA method to detect oligomers is based on polyclonal antibody A11 which recognizes a common structure in soluble amyloid oligomers (FIGS. 8-10, and 12). (Kayed et al., Science 300: 486-491(2003)). The most sensitive ELISA format for detection of tau oligomers with A11 was found to be to use capture antibody MN1000, reporter antibody A11, and biotinylated anti-rabbit antibody for signal enhancement (FIG. 9). This assay was used to optimize the molar ratio of tau:RNA for oligomer formation (FIGS. 8 and 15), follow oligomer formation over time (FIG. 10), show tau oligomer formation with a range of RNAs (FIG. 12), and detect soluble tau oligomers in CSF (FIG. 11).

A third ELISA was used to show biological relevance of the tau oligomers formed in this assay to tau in AD and is based on monoclonal antibodies DC11 used for capture and biotinylated MN1000 for reporter. The antibody DC11 recognizes a conformational epitope in tau specific for AD (Vechterova et al., Neuroreport 1:87-91 (2003)). Thus, recognition of the oligomer formed in vitro by this antibody indicates that the structure of tau in the oligomer has changed to a disease-associated conformation. This assay was used to follow RNA-facilitated oligomer formation over time, optimize the molar ratio of tau:RNA for oligomer formation (FIG. 15), and detect soluble tau oligomers in CSF.

### Example 4

Tau Oligomer Size is Dependent on RNA Length

To investigate whether oligomer size can be controlled by choice of RNA, soluble tau oligomers were formed rapidly under physiological temperature and pH as described in Example 1 using RNAs of different lengths RQT157 (157 bases) and RQ11+12 (197 bases). For oligomer formation 12 μM tau441 and 2 μM of either RNA were incubated in a total volume of 25 μL 0.05 M Tris-HCl, pH 7.4 for 30 minutes at 37° C.

The reaction products were immediately purified using size-exclusion chromatography (described above). One ml fractions were collected every 6 minutes and analyzed by modified ELISA using anti-tau MN1000 as capture antibodies, and biotin-MN1000 as reporter antibodies. A distinct peak of 300 kDa was identified in the control reaction (FIG. 2). Although 300 kDa is six times the molecular weight of tau441 this peak may actually represent dimers because the apparent size of this complex is expected to be larger than the calculated molecular weight due to the highly unfolded structure of tau protein. Supporting this notion, standard ELISA using MN1010 antibody for capture and MN1000 antibody for reporter showed that there was a major peak at about 150 kDa, apparently representing monomers. Incubation of tau441 with RQ11+12 RNA produced a major peak at 600 kDa and a smaller peak at 750 kDa. Incubation of tau441 with RQT157 RNA produced two major peaks of 400 kDa and 600 kDa. The size range of the oligomers formed with the larger RNA was greater than the size range of the oligomers formed with the smaller RNA. Thus, the size of a polyanion may be used to modulate the sizes of tau oligomers formed.

### Example 5

Tau Oligomer Formation Using Other Polyanions

Heparin:

Heparin (approx 7-10 kDa) was used to facilitate tau oligomer formation (FIG. 13), by incubating tau412 (20 pmole) with 20 pmole and 4 pmoles of heparin in 50 mM Tris-HCl, pH=7.4 for one hour at 37° C. Oligomer formation was determined by modified ELISA (described above). A 1:1 tau:heparin ratio was found to work better than a 5:1 ratio, as analyzed by the modified ELISA. The optimal tau:polyanion ratio depends on the nature of the polyanion. Heparin glycosaminoglycans (GAGs) usually contain an average of ˜2.4 sulfates per repeating disaccharide unit (Höök M, et al., Biochem. J 137:33-43, (1974)), whereas nucleic acids have one phosphate per base. The different backbone structure and charge distribution compared to nucleic acids may relate to the greater concentration needed of this polyanion to facilitate the same amount of tau to form oligomers.

DNA:

Tau412 oligomer formation was facilitated by different DNAs as determined by modified ELISA, showing the general efficacy of this polyanion to facilitate tau oligomer formation (FIG. 16). Sheared salmon sperm DNA (Shsalsp) composed of fragments of 300-600 bases of single and double stranded DNA, Calf Thymus DNA (CTD) composed of fragments less than 2000 bases of single and double stranded DNA, and Polydeoxythymidine (PDT) single strand DNA with a size distribution of 460-2600 nucleotides all enhanced tau412 oligomer formation.

Tau412 (20 pmoles in 50 uL) was incubated with 5 uL of RQ11+12 (4 pmoles) and also with 5 uL of the following DNA solutions:
Sheared salmon sperm DNA (Shsalsp): A stock solution was made by diluting 10 uL/1 mL tris HCl. Working dilutions: 1 to 250 of stock solution and: 1 to 10 of dilution 1.
Calf Thymus DNA (CTD): A stock solution was made by diluting 10 uL/1 mL tris HCl. Working dilution: 1 to 2500.
Polydeoxythymidine (poly dT) (PDT): 5 units/vial. dissolved in 300 uL Tris-HCL. Working dilutions: 1 to 250 of stock solution and: 1 to 10 of dilution 1.

### Example 6

Additional Polyanions to be Used in Oligomer Formation

1) Polyanionic polysaccharides:

Heparin, a sulfated glycosaminoglycan was effective in facilitating tau oligomer formation, thus, other sulfated polysaccharides may have similar function. For example, dextran sulfate and pentosan polysulfate have an average of 3-4 sulfates per repeating unit, are commercially available, reasonably priced and should be straightforward to test.
2) Poly-Glutamate: The polyanionic polypeptide has a different backbone and charge density than nucleic acids or sulfated glycosaminoglycan, therefore a range of concentrations and ratios with tau would need to be tested.
3) Anionic detergents: Anionic detergents have been used to facilitate rapid tau filimentation (Chirita C. N. et al., J. Bio. Chem., 278(28):25644-50 (2003)). It is postulated that, at the correct ratio of tau:detergent, oligomer formation can be facilitated by the polyanionic micelles the detergents form. Examples are alkyl detergents, C₁₂H₃₇SO₄Na, C₁₈H₃₇SO₄Na and C₂₀H₃₇SO₄Na.
4) Synthetic polyanions: The following are examples of polyanions which have potential for use in oligomer formation. This list is meant to be exemplary and is not exhaustive of all synthetic polyanions which may be used:
   polyanethole sulfonic acid
   polysulfonyl sulfone
   polyethylene sulfonate
   poly(acrylic acid)
   poly(methacrylic acid)
   sodium poly(styrene sulphonate)
   polyvinyl sulfonate

These polymers have limited anionic structural similarities to heparin sulfate and may have similar efficacy. Several concentrations and ratios would need to be tested.
5) Suramin (polysulfonated napthylurea):

Suramin inhibited amyloid fibril formation of human islet amyloid polypeptide at low concentrations, but stimulated the process at high concentrations. The minimal amyloid-forming fragment of human islet amyloid polypeptide is a glycolipid-binding domain supporting the role of aromatic pi-pi and CH-pi stacking interactions in the molecular control of the amyloidogenesis process. (Levy M, et al., FEBS J. 273(24):5724-35 (2006)). The polyaromatic structure and the anionic charge of suramin may both be involved in interactions with tau, therefore, a range of concentrations and ratios of tau:suramin will need to be tested.

What is claimed is:

1. A method of mimicking part of a pathway of an amyloid protein disease comprising incubating a mixture of an effective amount of a cationic amyloid protein and an effective amount of a polyanion, under physiological conditions, for a time period of about 10 minutes to about 120 minutes, to generate a stable soluble oligomer of the cationic amyloid protein that is indicative of the amyloid protein disease.

2. The method of claim 1, wherein the physiological conditions include a temperature of about 37° C. and a pH of about 7.4.

3. The method of claim 1, wherein the effective amount of the cationic amyloid protein is from about 40 nM to about 4 uM.

4. The method of claim 1, wherein the cationic amyloid protein is a tau protein.

5. The method of claim 4, wherein the polyanion is an RNA having a length of from about 10 nucleotides to about 10,000 nucleotides.

6. The method of claim 5, wherein the RNA is RQ11+12.

7. The method of claim 5, wherein the mixture comprises a ratio of from about 2:1 to about 20:1 of tau protein to the RNA.

8. The method of claim 1, wherein the soluble oligomer comprises about 3 to about 20 subunits of the cationic amyloid protein.

9. The method of claim 1, wherein the time period is about 30 to about 60 minutes.

10. The method of claim 1, wherein the polyanion is a DNA having a length of from about 10 nucleotides to about 10,000 nucleotides.

11. The method of claim 10, wherein the DNA is single stranded.

12. The method of claim 10, wherein the DNA is double stranded.

13. The method of claim 1, wherein the polyanion has a molecular weight of about 2,000 Daltons to about 125,000 Daltons.

14. The method of claim 13, wherein the polyanion is a proteoglycan.

15. The method of claim 13, wherein the polyanion is a sulfated polysaccharide.

16. The method of claim 14, wherein the proteoglycan is heparin.

17. The method of claim 13, wherein the polyanion is a synthetic polyanion.

18. The method of claim 17, wherein the synthetic polyanion has a linear structure.

19. The method of claim 17, wherein the synthetic polyanion has a branched structure.

20. The method of claim 17, wherein the synthetic polyanion has a circular structure.

21. A soluble oligomer of a cationic amyloid protein prepared by the process of incubating a mixture of an effective amount of a cationic amyloid protein and an effective amount of a polyanion, under physiological conditions, for a time period of about 10 minutes to about 120 minutes.

22. An antibody generated using the soluble oligomer of claim 21.

23. The antibody of claim 22, wherein the antibody is a monoclonal antibody.

24. A method for evaluating the ability of a therapeutic to affect an amyloid disease comprising:
(i) incubating a mixture of a cationic amyloid protein, a polyanion, under physiological conditions;
(ii) applying a therapeutic to be evaluated to the mixture;
(iii) analyzing the mixture to detect the presence or absence of a soluble oligomer of the cationic amyloid protein;
whereby the absence of a soluble oligomer of the cationic amyloid protein indicates the therapeutic is able to inhibit oligomer formation or disaggregate oligomer of the cationic amyloid protein, thereby indicating potential ability of the therapeutic to affect an amyloid disease.

25. The method of claim 24, wherein the physiological conditions are a temperature of about 37° C. and a pH of about 7.4.

26. The method of claim 24, wherein the mixture is incubated for about 10 minutes to about 120 minutes.

27. The method of claim 24, wherein the cationic amyloid protein used in the mixture is about 40 nM to about 4 μM.

28. The method of claim 24, wherein the cationic amyloid protein is a tau protein.

29. The method of claim 24, wherein the polyanion is an RNA having a length of from about 10 nucleotides to about 10,000 nucleotides.

30. The method of claim 29, wherein the RNA is RQ11+12.

31. The method of claim 29, wherein the mixture comprises a ratio of from about 2:1 to about 20:1 of cationic amyloid protein to RNA.

32. The method of claim 24, wherein the soluble oligomer comprises about 3 to about 20 subunits of the cationic amyloid protein.

33. The method of claim 24, wherein the polyanion is a synthetic polyanion having a molecular weight of about 2,000 Daltons to about 125,000 Daltons.

34. The method of claim 33, wherein the synthetic polyanion has a linear structure.

35. The method of claim 33, wherein the synthetic polyanion has a branched structure.

36. The method of claim 33, wherein the synthetic polyanion has a circular structure.

37. The method of claim 24, wherein the mixture is analyzed using an immunological assay.

38. The method of claim 37, wherein the immunological assay is a standard ELISA.

39. The method of claim 37, wherein the immunological assay is a modified ELISA that uses the same antibody to capture and to report the soluble oligomer of the cationic amyloid protein.

40. The method of claim 37, wherein an A11 antibody is used in the immunological assay.

41. The method of claim 37, wherein a DC11 antibody is used in the immunological assay.

42. An assay for evaluating the ability of a therapeutic to affect amyloid diseases, wherein the compound is evaluated using the method of claim 24.

43. A method for identifying a therapeutic which modulates the formation of soluble tau oligomers comprising:
(i) incubating a mixture of a tau isoform and a polyanion under physiological conditions;
(ii) applying a therapeutic to the mixture;
(iii) analyzing the mixture to detect the presence or absence of a soluble tau oligomer; and
(iv) correlating the presence or absence of a soluble tau oligomer in the mixture to modulation of the formation of soluble tau oligomers by the therapeutic, whereby the absence of soluble tau oligomer indicates the therapeutic modulates the formation of soluble tau oligomers.

44. An assay for identifying a therapeutic which modulates the formation of soluble tau oligomers, wherein the therapeutic is identified using the method of claim 43.

45. A method of preparing a soluble tau oligomer that is stable under physiological conditions comprising incubating a mixture of a tau isoform with a polyanion, in a ratio from about 2:1 to about 20:1 of the tau isoform to the polyanion, under physiological conditions for about 30 minutes to about 60 minutes.

46. A method of generating a stable, soluble oligomer of a cationic amyloid protein of a specified number of subunits comprising:
i) choosing a polyanion of appropriate size and charge distribution;
ii) mixing an effective amount of the polyanion with an effective amount of a cationic amyloid protein to create a mixture under physiological conditions;
iii) allowing sufficient time for the polyanion and cationic amyloid protein to form stable and soluble oligomers of the cationic amyloid protein of specified number of subunits; such that the effective amounts of the polyanion and cationic amyloid protein are determined so that the cationic amyloid protein will be completely depleted after allowing sufficient time for formation of the stable and soluble oligomers, thereby trapping the stable and soluble oligomers at the specified number of subunits due to the size and charge distribution of the chosen polyanion.

47. The method of claim 46, wherein the cationic amyloid protein is tau protein.

48. The method of claim 46, wherein the polyanion is a nucleic acid having from about 10 to about 10,000 nucleotides.

49. The method of claim 48, wherein the nucleic acid is RNA.

50. The method of claim 49, wherein the RNA is RQ11+12.

51. The method of claim 46, wherein the polyanion has a molecular weight of about 2,000 to about 125,000 Daltons.

52. The method of claim 51, wherein the polyanion is a synthetic polyanion.

53. The method of claim 51, wherein the polyanion is heparin.

54. The method of claim 46, wherein the effective amount of cationic amyloid protein is about 40 nM to about 4 μM.

55. The method of claim 46, wherein the effective amount of polyanion and the effective amount of cationic amyloid protein are in a ratio of from about 2:1 to about 20:1 of cationic amyloid protein to polyanion.

57. The method of claim 46, wherein the specified number of subunits is from 3 to 20.
